(19) United States
(12) Patent Application Publication
(10) Pub. No.: US 2018/0161372 A1
(43) Pub. Date: Jun. 14, 2018

(54) COMPOSITION FOR TREATING BRAIN LESIONS

(71) Applicants: Denis BARRITAULT, Paris (FR); ORGANES TISSUS REGENERATION REPARATION REMPLACEMENT - OTR3, Paris (FR); CENTRE NATIONAL DE LA RECHERCHE SCIENTIFIQUE, Paris (FR); UNIVERSITE DE CAEN BASSE-NORMANDIE, Caen (FR)

(72) Inventors: Myriam BERNAUDIN, Bernieres Sur Mer (FR); Omar TOUZANI, Villy-Bocage (FR); Jérôme TOUTAIN, Ondefontaine (FR); Marie-Sophie QUITTET, Le Havre (FR); Denis BARRITAULT, Paris (FR)

(21) Appl. No.: 15/577,634

(22) PCT Filed: May 26, 2016
(86) PCT No.: PCT/EP2016/061905

§ 371 Date: Nov. 28, 2017

(30) Foreign Application Priority Data
May 28, 2015 (EP) 15305806.0

(51) Int. Cl. A61K 35/28; A61K 31/737; A61P 25/00

(52) U.S. Cl. CPC A61K 35/28; A61P 25/00; A61K 31/737

(57) ABSTRACT

Some embodiments are directed to a pharmaceutical composition which includes a biocompatible polymer and a eukaryotic cell to be used as a drug for the prevention and/or treatment of tissue lesions of the central nervous system caused by cerebral vascular ischaemia. Some embodiments are also directed to a pharmaceutical kit which includes a biocompatible polymer and a eukaryotic cell for the prevention and/or treatment of tissue lesions of the central nervous system caused by cerebral vascular ischaemia. Some other embodiments can be used in particular in the human and veterinary pharmaceutical fields.

### CROSS REFERENCE TO RELATED APPLICATIONS

This application is a national phase filing under 35 C.F.R. § 371 of and claims priority to PCT Patent Application No. PCT/EP2016/061905, filed on May 26, 2016, which claims the priority benefit under 35 U.S.C. § 119 of European Patent Application No 15305806.0, filed on May 28, 2015, the contents of each of which are hereby incorporated in their entireties by reference.

### BACKGROUND

Some embodiments are directed to a pharmaceutical composition for use as a medicament for the prevention and/or treatment of tissue lesions of the central nervous system caused by cerebral vascular ischemia.

Some embodiments are also directed to a pharmaceutical kit for the prevention and/or treatment of tissue lesions of the central nervous system caused by cerebral vascular ischemia.

Some embodiments can be used in particular in the human and veterinary pharmaceutical fields.

In the description below, the references between parentheses ( ) refer to the list of references presented at the end of the text.

Strokes (CVAs) represent the primary cause of morbidity and the third cause of mortality in human beings in industrialized countries. This pathological condition takes a very heavy toll: 10% to 12% of all or most deaths after the age of 65 and also physical, cognitive or psychological after-effects in more than half of victims. According to the WHO, 15 million individuals suffer a stroke throughout the world each year. Among these, 5 million die and 5 million others are disabled for life. In Europe, the number of deaths caused by stroke is estimated at approximately 650 000 each year. Consequently, the socioeconomic repercussions of strokes are very considerable (5.3 billion euros in 2007 in France (Chevreul et al., 2013).

Stroke is defined as the decrease in the blood supply in a given area of the brain. There are two types of stroke: hemorrhagic event, which corresponds to blood leaking from the vascular compartment into the cellular compartment as a result of the rupturing of a blood vessel, and the ischemic type, to which 80% of patients suffering from stroke fall victim. The latter is due to the decrease in blood flow caused by an embolism corresponding to a clot which is thought to become detached from the periphery and is thought to be carried to the cerebral artery, or by an atherosclerosis plaque which ultimately totally occludes the lumen of the vessel. The artery most commonly involved in this occlusion is the Sylvian artery or middle cerebral artery (MCA). It is an artery that irrigates a major part of the cerebral hemisphere and the occlusion of which causes a significant sensorimotor or cognitive handicap (hemiplegia, hemiparesis, agnosia, memory deficit, etc.) (Cramer, 2008; Jaillard et al., 2009).

Treatment of Ischemic Stroke

Cerebral ischemia can be defined as an inadequate blood supply in relation to metabolic demand. This is caused by a decrease in cerebral blood flow which may be transient or long-lasting. The cerebral lesion which accompanies focal ischemia generally can include or can consist of a severely affected center and a peripheral zone of which the viability is precarious; this zone, called penumbra, can be recruited by the necrosis process unless a therapeutic intervention is instituted in time (Touzani et al., 2001). The ischemic penumbra thus represents the target of any therapeutic intervention during the acute phase of cerebral ischemia.

Despite the considerable public health problem represented by ischemic stroke, the therapeutic arsenal for combatting the latter is small. Currently, only thrombolysis with t-PA (tissue plasminogen activator) is approved by the health authorities. However, the use of t-PA is restricted by virtue of its small therapeutic window, namely from 3 to 4.5 h after the occurrence of the stroke, and the numerous contraindications that are associated therewith, linked to the risks of cerebral hemorrhage (absence of blood-thinning treatment, absence of (cerebral or cardiac) ischemic event in the previous 3 months, absence of gastrointestinal or urinary hemorrhage in the last 21 days, absence of bleeding, arterial blood pressure <185/110 mmHg systolic/diastolic, etc.). According to Lees and collaborators (2010), the administration of rt-PA after a period of more than 4 h30 causes a risk of cerebral hemorrhage that is significantly higher than in untreated patients, and is associated with an unfavorable benefit-risk balance (Lees et al., 2010). Thus, it is estimated that only 3% to 5% of patients can have recourse to this treatment (Adeoye et al., 2011) and despite the strict selection of patients, it is evaluated that 13% of them will develop a cerebral hemorrhage following the administration of rt-PA.

There is therefore a real need to find a new composition/medicament that overcomes these faults, drawbacks and obstacles, in particular for a composition which makes it possible in particular to treat/stop a stroke, which has in particular a broad treatment window and/or which decreases/eliminates the side effects due to the treatment.

Apart from thrombolysis using t-PA, numerous investigations in animals having shown a possible efficacy of several therapeutic strategies aimed at protecting the neurons against ischemia (Kaur et al., 2013). Among these strategies, mention may be made of calcium channel blockage, inhibition of oxidative stress, GABA A receptor stimulation, inhibition of NMDA and AMPA receptors. However, in human clinical practice, success of these therapeutic interventions has not been found (Kaur et al., 2013).

There is therefore a real need to find a new composition/medicament which makes it possible to treat stroke and the tissue consequences thereof.

Given the dramatic failures of several clinical trials having tested therapeutic interventions for neuroprotection after a stroke in human beings, numerous authors are turning to the development of brain repair strategies applicable during the subacute or chronic phase of the pathological condition. These strategies can include or can consist of the provision of neurotrophic factors or of the transplantation of stem cells in order to promote functional recovery.

Stem Cells and Cerebral Ischemia

Several types of stem cells have been tested in animals subjected to cerebral ischemia. Among these, mention may be made of embryonic stem cells (ESCs), induced pluripotent stem cells (iPSCs), neural stem cells (NSCs) and mesenchymal stem cells (MSCs) (for review, see Hao et al., 2014). Although ESCs and iPSCs have shown beneficial effects in animals after ischemia, their problems of availability (for ESCs) and their capacity to transform into tumors limit, for the moment, their use in human beings. Indeed, it has been demonstrated that these cells are capable of being responsible for the generation of tumors after injection.

Neural stem cells (NSCs) are found fetal tissue, neonatal tissue, in young individuals but also in adults. The neuroblastic stem cell niches in human beings and in animals are the subventricular zone (SVZ) and the subgranular zone of the dentate gyrus (Seri et al., 2006). Although these cells are already oriented in terms of their differentiation, they are termed stem cells because they are capable, in the context of particular differentiation protocols, of differentiating into hippocampal neurons, into cortical neurons or else into motoneurons or interneurons. There are many studies in the literature which have shown beneficial effects of NSC transplantation after cerebral ischemia, for example as described in the document Hao and collaborators, 2014. For example, the administration of NSCs in an ischemic cortical lesion or in its periphery promotes the production of neuroblasts in the SVZ. Stimulation of the dendritic arborization and also of axonal growth correlated with an increase in functional recovery is observed after their administration in rats (Andres et al., 2011). However, several constraints limit the use of these cells in clinical practice. This is because the isolation of these cells from fetuses is made difficult by the ethical constraints. Another source of NSCs would be cerebral biopsy of the SVZ, which can only be carried out post-mortem in the case of ischemia, greatly limiting the amount of resources and rendering difficult the recourse to autotransplantation in the patient.

Another approach explored is the use of other stem cells that are more accessible, such as mesenchymal stem cells (MSCs). These cells were identified for the first time by Friedenstein and collaborators in 1970, who characterized these cells as adhering to plastic and rare (Friedenstein et al., 1970). Several sources have been identified and used, including mainly bone marrow, but also dental pulp (Yalvac et al., 2009; Yamagata et al., 2013), hair follicle (Wang et al., 2013), placenta (In't Anker et al., 2004) or umbilical cord (Erices et al., 2000; Kranz et al., 2010).

Like all or most stem cells, MSCs can differentiate into specialized cells and self-renew. MSCs are capable of differentiating, in vitro, into several cell types, and, in a suitable environment and under suitable conditions, they are capable of differentiating to a non-mesenchymal phenotype such as the neuronal or cardiomyocyte phenotype (Esneault et al., 2008; Toma et al., 2002). The ease of access to and of extraction of these cells from the bone marrow and their easy and rapid multiplication could make it possible to perform autologous transplants capable of limiting the use of immunosuppressor treatments that are difficult for patients to tolerate. Moreover, mesenchymal stem cells do not express the type II (HLA-DR or HLA type II) major histocompatibility complex (MHC) and express only small amounts of type I (HLA-ABC or HLA type I) MHC on the membrane. In addition, Di Nicola and collaborators in 2002 demonstrated a decrease in T lymphocyte proliferation under conditions of coculture with MSCs, this being in a dose-dependent and reversible manner (Di Nicola, 2002). In addition to T lymphocytes, MSCs can have an anti-inflammatory action on other cells of inflammation, such as Natural Killer cells, dendritic cells or macrophages (Aggarwal & Pittenger, 2005; Eckert et al., 2013). The clinical trials carried out in the context of cardiac, nervous or else immune diseases have not, a priori, demonstrated serious adverse effects following an administration of MSCs (Malgieri et al., 2010).

In cerebral ischemia, a post-ischemic functional benefit obtained after the administration of MSCs has been demonstrated by preclinical studies and some clinical studies as summarized in Hao et al., 2014, and Kaladka and Muir, 2014.

With regard to the clinical studies, Bang and collaborators (2005) administered, for the first time, MSCs to patients having undergone cerebral ischemia. This first study was carried out on few patients (5 compared with 25 controls) but demonstrated an absence of tumor development and a possible feasibility of the autologous administration of MSCs in patients having suffered a stroke. An improvement in post-ischemic functional recovery of the treated patients was observed by the authors 3 to 6 months post-treatment. These results were confirmed in 2010 via an IV administration of MSCs in 16 patients suffering from stroke. In particular, a relative decrease in the mortality of the treated patients was observed. A beneficial effect of the treatment on functional recovery was shown over an observation period of 5 years by way of mRS (Lee et al., 2010). Since then, other studies have made it possible to strengthen the notion of feasibility and safety of this new therapeutic strategy (Bhasin et al., 2011; Suárez-Monteagudo et al., 2009). Several phenomena explain the efficacy of MSCs, such as their paracrine property regarding neurogenic or angiogenic growth factors (FGF2, NGFb, EGF, VEGF-A, IGF1, BDNF).

However, despite the numerous advantages that they provide, MSCs have a very limited survival after administration into an ischemic zone. Indeed, 99% of the cells die during the first 24 hours and, according to Toma and collaborators (2002), only 0.5% of MSCs implanted into an ischemic environment survive 4 days after the implantation. Several phenomena explain this cell loss (Toma et al., 2002). Indeed, inflammation, hypoxia, anoikis (absence of support) or the pro-apoptotic factors present in the surrounding medium induce the triggering of apoptosis. Furthermore, since cerebral ischemia is characterized by a reduction in cerebral blood flow, the grafted cells therefore lack energy substrates essential to their survival. The neutrophils and macrophages recruited into the ischemic zone will, in addition, produce oxygenated radicals, for which Song and collaborators (Song, Cha, et al., 2010) have demonstrated, in the case of cardiac ischemia, the harmful effect on the attachment of mesenchymal stem cells. As it happens, the adhesion of cells to the extracellular matrix of the surrounding medium via integrin proteins induces a positive signal in the cell and a repression of apoptosis, whereas the reverse phenomenon occurs in the case of a lack of support (Song, Song, et al., 2010). Furthermore, following ischemia, the extracellular matrix is destroyed by metalloproteases and the persistence of these metalloproteases limits the reconstruction of the ECM. According to Toma and collaborators, one of the major factors in the death of the injected cells is thought to be the absence of growth support, inducing anoikis. The phenomenon is juxtaposed with the presence of free radicals which further restrict the incorporation of the graft into the host tissue. In addition, tissue regeneration in the case of ischemia is greatly dependent on the vascularization of the healing zone.

There is also a real need to find a new composition which overcomes the faults, drawbacks and obstacles of the prior art, in particular for a composition which makes it possible in particular to treat/stop a stroke, to treat the consequences/effects of a stroke, to reduce the cost and to improve the therapeutic/dosage regimen scheme of stroke treatment.

### SUMMARY

An objective of some embodiments is precisely to meet these needs by providing a pharmaceutical composition for use as a medicament for the prevention and/or treatment of tissue lesions of the central nervous system caused by a cerebral hypoxic pathological condition, the composition including
a biocompatible polymer of general formula (I) below

AaXxYy (I)
wherein:
A represents a monomer,
X represents an —R₁COOR₂ or —R₉(C═O)R₁₀ group,
Y represents an O- or N-sulfonate group which corresponds to one of
the following formulae —R₃OSO₃R₄, —R₅NSO₃R₆, —R₇SO₃R₈
   wherein:
R₁, R₃, R₅ and R₉ independently represent an aliphatic hydrocarbon-based chain which is optionally branched and/or unsaturated and which optionally contains one or more aromatic rings,
R₂, R₄, R₆ and R₈ independently represent a hydrogen atom or a cation,
R₇ and R₁₀ independently represent a bond, or an aliphatic hydrocarbon-based chain which is optionally branched and/or unsaturated,
“a” represents the number of monomers,
“x” represents the degree of substitution of the monomers A by groups X,
“y” represents the degree of substitution of the monomers A by groups Y, and
a eukaryotic cell.

In the present document, the term “tissue lesions of the central nervous system” is intended to mean any tissue lesions that may appear in the central nervous system. It may for example be a tissue lesion due to a physical impact, for example linked to a trauma, a tissue lesion due to an ischemic shock, for example due to a transient and/or long-lasting decrease in cerebral blood flow linked for example to a vascular occlusion, a vascular hemorrhage or else a hypoxic shock.

In the present document, the term “cerebral hypoxic pathological condition” is intended to mean any pathological condition and/or event capable of causing a decrease in oxygen supply to the brain.

It may for example be a vascular event, a cardiac arrest, hypotension, one or more complications associated with anesthesia during a procedure, suffocation, carbon monoxide poisoning, drowning, inhalation of carbon monoxide or of smoke, brain lesions, strangulation, an asthma attack, a trauma, a tissue lesion due to an ischemic shock, perinatal hypoxia, etc.

In the present document, the term “monomer” is intended to mean for example a monomer chosen from the group including sugars, esters, alcohols, amino acids or nucleotides.

In the present document, the monomers A constituting the basic elements of the polymers of formula I can be identical or different.

In the present document, the linking of monomers can make it possible to form a polymer backbone, for example a polymer backbone of polyester, polyalcohol or polysaccharide nature, or of the nucleic acid or protein type.

In the present document, among the polyesters, there may for example be copolymers from biosynthesis or chemical synthesis, for example aliphatic polyesters, or copolymers of natural origin, for example polyhydroxyalkanoates.

In the present document, the polysaccharides and derivatives thereof may be of bacterial, animal, fungal and/or plant origin. They may for example be single-chain polysaccharides, for example polyglucoses, for example dextran, cellulose, beta-glucan, or other monomers including more complex units, for example xanthans, for example glucose, mannose and glucuronic acid, or else glucuronans and glucoglucuronan.

In the present document, the polysaccharides of plant origin may be single-chain, for example cellulose (glucose), pectins (galacturonic acid), fucans, or starch, or may be more complex, for instance alginates (galuronic and mannuronic acid).

In the present document, the polysaccharides of fungal origin may for example be steroglucan.

In the present document, the polysaccharides of animal origin may for example be chitins or chitosan (glucosamine).

The number of monomers A defined in formula (I) by “a” may be such that the weight of the polymers of formula (I) is greater than approximately 2000 daltons (which corresponds to 10 glucose monomers). The number of monomers A defined in formula (I) by “a” may be such that the weight of the polymers of formula (I) is less than approximately 2 000 000 daltons (which corresponds to 10 000 glucose monomers). Advantageously, the weight of the polymers of formula (I) may be from 2 to 100 kdaltons.

In the present document, in the —R₁COOR₂ group representing X, R₁ may be a C₁ to C₆ alkyl, for example a methyl, an ethyl, a butyl, a propyl or a pentyl, possibly a methyl group, and R₂ may be a bond, a C₁ to C₆ alkyl, for example a methyl, an ethyl, a butyl, a propyl or a pentyl, or an R₂₁R₂₂ group in which R₂₁ is an anion and R₂₂ a cation chosen from the group of alkali metals.

Possibly, the group X is the group of formula —R₁COOR₂ in which R₁ is a methyl group —CH₂— and R₂ is an R₂₁R₂₂ group in which R₂₁ is an anion and R₂₂ a cation chosen from the group of alkali metals, possibly the group X is a group of formula —CH₂—COO⁻.

In the present document, in the —R₉(C═O)R₁₀ group representing X, R₉ may be a C₁ to C₆ alkyl, for example a methyl, an ethyl, a butyl, a propyl or a pentyl, possibly a methyl group, and R₁₀ may be a bond, or a C₁ to C₆ alkyl, for example a methyl, an ethyl, a butyl, a propyl, a pentyl or a hexyl.

In the present document, in the group corresponding to one of the following formulae —R₃OSO₃R₄, —R₅NSO₃R₆ and —R₇SO₃R₈ and representing the group Y, R₃ may be a bond, a C₁ to C₆ alkyl, for example a methyl, an ethyl, a butyl, a propyl or a pentyl, possibly a methyl group, R₅ may be a bond, a C₁ to C₆ alkyl, for example a methyl, an ethyl, a butyl, a propyl or a pentyl, possibly a methyl group, R₇ may be a bond, a C₁ to C₆ alkyl, for example a methyl, an ethyl, a butyl, a propyl or a pentyl, possibly a methyl group, and R₄, R₆ and R₈ may independently be a hydrogen atom or a cation M⁺, for example M⁺ may be an alkali metal.

Possibly, the group Y is the group of formula R₇SO₃R₈ in which R₇ is a bond and R₈ is an alkali metal chosen from the group including sodium, potassium, rubidium and cesium. Possibly, the group Y is an —SO₃⁻Na⁺group.

The degree of substitution of all or most of the monomers A by the groups Y defined in general formula (I) by “y” may be from 30% to 150%, and possibly about 100%.

In the present document, in the definition of the degrees of substitution above, the term “a degree of substitution “x” of 100%”, is intended to mean the fact that each monomer A of the polymer of some embodiments statistically contains a group X. Likewise, the term “a degree of substitution “y” of 100%” is intended to mean the fact that each monomer of the polymer of some embodiments statistically contains a group Y. The degrees of substitution greater than 100% reflect the fact that each monomer statistically bears more than one group of the type in question; conversely, the degrees of substitution of less than 100% reflect the fact that each monomer statistically bears less than one group of the type in question.

The polymers may also include functional chemical groups, denoted Z, different than X and Y.

In the present document, the groups Z may be identical or different, and may independently be chosen from the group including amino acids, fatty acids, fatty alcohols, ceramides, or mixtures thereof, or targeting nucleotide sequences.

The groups Z may also represent active agents, which may be identical or different. They may for example be therapeutic agents, diagnostic agents, an anti-inflammatory, an antimicrobial, an antibiotic, a growth factor, an enzyme.

In the present document, the group Z may advantageously be a saturated or unsaturated fatty acid. It may for example be a fatty acid chosen from the group including acetic acid, caprylic acid, capric acid, lauric acid, myristic acid, palmitic acid, stearic acid, arachidic acid, behenic acid, lignoceric acid, cerotic acid, myristoleic acid, palmitoleic acid, sapienic acid, oleic acid, elaidic acid, trans-vaccenic acid, linoleic acid, linolelaidic acid, α-linolenic acid, γ-linolenic acid, dihomo-γ-linolenic acid, arachidonic acid, eicosapentaenoic acid, clupanodonic acid or docosahexaenoic acid. Possibly, the fatty acid is acetic acid.

In the present document, the group Z may advantageously be an amino acid of the L or D series chosen from the group including alanine, asparagine, an aromatic chain, for example tyrosine, phenylalanine, tryptophan, thyroxine or histidine.

Advantageously, the groups Z may confer additional biological or physiochemical properties on the polymers. For example, the groups Z may increase the solubility or the lipophilicity of the polymer, enabling for example better tissue diffusion or penetration, for example the increase in amphiphilicity can enable a facilitation of the crossing of the blood-brain barrier.

Polymers in which Z is present correspond to formula II below:

AaXxYyZz

wherein A, X, Y, a, x and y are as defined above and z represents the degree of substitution by groups Z.

In the present document, the degree of substitution by groups Z represented by “z” can be from 0% to 50%, possibly equal to 30%.

The groups X, Y and Z can be independently bonded to the monomer A and/or independently bonded to one another. When at least one of the groups X, Y and Z is independently bonded to a group X, Y and Z different than the first, one of the groups X, Y or Z is bonded to the monomer A.

Thus, the groups Z can be covalently bonded directly to the monomers A or covalenty bonded to the groups X and/or Y.

In the present document, the composition can include a concentration of 0.01 microgram to 100 mg by weight of biocompatible polymer relative to the total weight of the composition. For example, the composition can include from 10 micrograms to 10 milligrams by weight relative to the total weight of the composition.

In the present document, the concentration of the biocompatible polymer in the composition and/or administration dosage regimen of the composition can depend on the route of administration envisioned for the composition according to some embodiments.

For example, for intracranial administration, it may be a single administration of 1 to 5 ml, or an administration by minipump, for example over several days. For example, the composition can include a concentration of 0.001 to 1 mg·ml⁻¹ of biocompatible polymer.

In the present document, the term “eukaryotic cell” is intended to mean any eukaryotic cell known to those with ordinary skill in the art. It can for example be a mammalian eukaryotic cell, for example an animal or human eukaryotic cell. It can for example be any eukaryotic cell regardless of its stage of differentiation, for example a cell chosen from the group including adult or embryonic eukaryotic cells, embryonic stem cells, and adult stem cells. It can for example be eukaryotic cells from umbilical cord blood, bone marrow cells, adipose tissue cells, mesenchymal cells.

It can also be a pluripotent or totipotent stem cell, or cells committed to differentiation pathways, for example mesenchymal stem cells. It can also be a pluripotent or totipotent stem cell with the exception of embryonic stem cells.

It can for example be a cell that is heterologous, homologous or autologous with respect to an individual. Possibly, the cells are autologous cells.

Advantageously, when the cells are autologous, the composition according to some embodiments may be possible for regulatory, safety, feasibility, efficiency and economic reasons.

Advantageously, when the cells are autologous, they are possibly isolated from the individual and used in the composition according to some embodiments and/or used in a treatment within 24 hours after removal and isolation without other additions. Advantageously, this single administration makes it possible to overcome and to comply with the regulatory requirements/constraints.

In the present document, the amount of cells included in the composition can be from 1 to 5×10⁷ cells.

In the present document, the term “pharmaceutical composition” is intended to mean any form of pharmaceutical composition known to those with ordinary skill in the art. In the present document, the pharmaceutical composition may for example be an injectable solution. It may for example be an injectable solution, for example for local or systemic injection, for example in physiological saline, in injectable glucose solution, in the presence of excipients, for example of dextrans, for example at concentrations known to those with ordinary skill in the art, for example from one milligram to a few milligrams per ml. The pharmaceutical composition may for example be a medicament intended for oral administration, chosen from the group including a liquid formulation, an oral effervescent dosage-regimen form, an oral powder, a multiparticle system, and an orodispersible galenic form.

For example, when the pharmaceutical composition is for oral administration, it may be in the form of a liquid formulation chosen form the group including a solution, a syrup, a suspension and an emulsion. When the pharmaceutical composition is in the form of an oral effervescent dosage-regimen form, it may be in a form chosen from the group including tablets, granules and powders. When the pharmaceutical composition is in the form of an oral powder or a multiparticulate system, it may be in a form chosen from the group including beads, granules, mini-tablets and the microgranules. When the pharmaceutical composition is in the form of an orodispersible dosage-regimen form, it may be in a form chosen from the group including orodispersible tablets, lyophilized wafers, thin films, a chewing tablet, a tablet, a capsule or a medical chewing gum.

According to some embodiments, the pharmaceutical composition can be a pharmaceutical composition for oral administration, for example buccal and/or sublingual administration, for example chosen from the group including buccal or sublingual tablets, lozenges, drops and a spray solution.

According to some embodiments, the pharmaceutical composition can be a pharmaceutical composition for topical, transdermal administration, for example chosen from the group including ointments, creams, gels, lotions, patches and foams.

According to some embodiments, the pharmaceutical composition can be a pharmaceutical composition for nasal administration, for example chosen from the group including nasal drops, a nasal spray and nasal powder.

According to some embodiments, the pharmaceutical composition can be a pharmaceutical composition for parenteral administration, for example subcutaneous, intramuscular, intravenous or intrathecal administration.

In the present document, the composition can be formulated and/or adjusted according to its administration. For example, for intravenous or intramuscular administration, the composition can be administered in order to deliver a dose of biocompatible polymer of from 0.1 to 5 mg per kilogram of body weight, or for oral administration the composition can be administered, for example, in 2 to 5 equal intakes per day in an amount of a daily total for example of from 15 to 500 mg of biocompatible polymer, or for intracranial administration the composition can include a concentration of from 0.001 to 1 mg·ml⁻¹ of biocompatible polymer, or for sublingual administration the composition can include a concentration of from 1 to 100 mg/ml of biocompatible polymer, or for aerial administration the composition can be administered in order to deliver a dose of from 0.1 to 5 mg of biocompatible polymer per kilogram of body weight, of the polymer.

The composition of some embodiments can also include at least one other active ingredient, particularly one other therapeutically active ingredient, for example for use which is simultaneous, separate or sequential over time depending on the galenic formulation used. This other ingredient can for example be an active ingredient used for example in the treatment of opportunistic diseases which can develop in a patient who has a tissue lesion of the central nervous system. It may also be pharmaceutical products known to those with ordinary skill in the art, for example antibiotics, anti-inflammatories, anticoagulants, growth factors, platelet extracts, neuroprotectors or else antidepressants, anticholesterols such as statins, etc.

In the present document, the administration of the biocompatible polymer and of the cell may be simultaneous, successive or concomitant.

According to some embodiments, at least one of the administrations can be carried out orally or by injection. The two administrations can be carried out in the same way or differently. For example, at least one of the administrations can be carried out orally or by injection. For example, the administration of the biocompatible polymer and of the cells can be carried out by injection, the administration of the biocompatible polymer can be carried out orally and the cells can be done by systemic injection or local injection. The administration can also depend on the site of the lesion.

According to some embodiments, the use of eukaryotic cells, in particular their administration, can be carried out within a period of from 5 minutes to 3 months, for example from 5 minutes to 1 week, possibly from 5 minutes to 24 hours, after the first administration of the biocompatible polymer.

According to some embodiments, the composition can for example be administered daily, twice-daily or weekly. It can for example be an administration once a day, twice a day or more.

According to some embodiments, the composition can for example be administered over a period of from 1 day to 3 months, for example for 2 months. For example, the composition can be administered over a period of 3 months with an administration frequency every 15 days.

According to some embodiments, the biopolymer can for example be administered over a period of from 1 day to 3 months, for example for 2 months, with for example a frequency of once a day, and the eukaryotic cell can be administered over an identical or different period, for example a period of from 1 day to 3 months, with a weekly frequency.

According to some embodiments, when the administration of the polymers and the administration of the cells are successive, the dosage regimen for each administration can be administration of the polymers followed by the administration of the cells. For example, the cells can be administered from 1 minute to 24 hours after the administration of the polymers, from 30 minutes to 12 hours after administration of the polymers, from 45 minutes to 6 hours after administration of the polymers, 1 hour after administration of the polymers.

Some embodiments also relate to a method for treating a patient having suffered cerebral ischemia, including, in any order, the following steps:

i. the administration of at least one biocompatible polymer, and

ii. the administration of at least one eukaryotic cell, wherein the administrations are concomitant, successive or alternating.

The biocompatible polymer is as defined above.

The eukaryotic cell is as defined above.

According to some embodiments, the patient can be any mammal. The patient can for example be an animal or a human being.

According to some embodiments, the eukaryotic cell administered can be a cell that is heterologous or homologous with respect to the patient.

According to some embodiments, the method and/or the route of administration of the biocompatible polymer can be as defined above.

According to some embodiments, the method and/or the route of administration of the cell can be as defined above.

According to some embodiments, the frequency of administration of the biocompatible polymer can be as defined above.

According to some embodiments, the frequency of administration of the eukaryotic cell can be as defined above.

According to some embodiments, when the administrations of the biocompatible polymers and of the cells are successive, the dosage regimen for each administration can be administration of the biocompatible polymers followed by the administration of the cells. For example, the cells can be administered from 1 minute to 48 hours after the administration of the biocompatible polymers, from 30 minutes to 12 hours after administration of the polymers, from 45 minutes to 6 hours after administration of the polymers, 1 hour after administration of the polymers.

Advantageously, the eukaryotic cell is a mesenchymal adult stem cell.

In other words, even though in the present description reference is made to a composition, it is clearly understood that each of the compounds of the composition can be administered concomitantly with the other compounds (for example in a single composition or in two compositions, each of these compositions including one or more of the abovementioned components, the method of administration of each of the compounds or composition(s) possibly being identical or different), or independently of one another, for example successively, for example independent administration of a biocompatible polymer, and independent administration of a eukaryotic cell, these administrations being carried out on one and the same patient, concomitantly or successively or in an alternating manner, in an order which is that mentioned above or another order. These various administrations can be carried out independently of one another or in a linked manner (composition or co-administration), by an identical or different method of administration (injection, ingestion, topical application, etc.), one or more times a day, for one or more days which may or may not be successive.

A subject of some embodiments is also a pharmaceutical kit for the prevention and/or treatment of tissue lesions of the central nervous system caused by cerebral vascular ischemia, including:

i. a biocompatible polymer, and

ii. at least one eukaryotic cell.

The biocompatible polymer is as defined above.

The eukaryotic cell is as defined above.

Some embodiments are also directed to the use of a pharmaceutical composition, including:

i. a biocompatible polymer, and

ii. at least one eukaryotic cell

for producing a medicament for the treatment of tissue lesions of the central nervous system caused by cerebral vascular ischemia.

The biocompatible polymer is as defined above.

The eukaryotic cell is as defined above.

In this embodiment, the term “medicament” is intended to mean a pharmaceutical composition as defined above.

The presently disclosed subject matter demonstrates, surprisingly and unexpectedly, that the composition according to some embodiments advantageously enables a significant decrease in ischemic lesions.

In addition, the presently disclosed subject matter also demonstrates that the composition according to some embodiments advantageously enables an early and long-lasting post-ischemic functional recovery.

The presently disclosed subject matter also demonstrates that the composition according to some embodiments advantageously enables an early improvement in neurological function and in sensorimotor performance after administration of the composition according to some embodiments.

Furthermore, the presently disclosed subject matter also demonstrates that the composition according to some embodiments advantageously makes it possible to limit/reduce the volume of infarction caused for example by a tissue lesion associated for example with a stroke.

In addition, the presently disclosed subject matter also demonstrates that the composition according to some embodiments advantageously makes it possible to protect and/or stimulate the regeneration of cerebral tissue exhibiting lesions associated for example with a stroke and/or radiotherapy treatment.

Other advantages may further emerge to those with ordinary skill in the art on reading the examples below, illustrated by the appended figures, given by way of illustration.

### BRIEF DESCRIPTION OF THE FIGURES

FIG. 1 represents a diagram of the experimental protocol aimed at studying the effects of a biocompatible polymer on brain damage and neurological deficits. In this figure, MCAo signifies middle cerebral artery occlusion, LP signifies limb placing test; NS signifies neurological score; OF signifies open field; MRI signifies magnetic resonance imaging.

FIG. 2 represents photographs of the central nervous system (brain) (FIG. 2 A) representing an infarction (area within dashed line) without application (1) or after application (2) of a biocompatible polymer after two days (D2) or fourteen days (D14) following the lesion-inducing ischemic event.

FIG. 2 B represents a diagram representing the volume of the lesion (y-axis) as a function of the day (x-axis) without (white bars) or with application of a biocompatible polymer (black bars).

FIG. 3 represents a diagram (FIG. 3 A) representing the results of the limb placing test (*repeated measure ANOVA p<0.05) as a function of the time after administration (solid triangles) or non-administration (empty triangles) of a biocompatible polymer. FIG. 3 B represents a bar diagram of the lateralization results evaluated using the corner test (* comparison of the mean to the reference value 0 p<0.05) at more or less three days after administration (solid bars) or non-administration (empty bars) of a biocompatible polymer. FIG. 3 C represents a bar diagram of the evaluation of the fine sensorimotor recovery using the adhesive withdrawal test (*p<0.05, one-way ANOVA) after 2 or 4 weeks after administration (solid bars) or non-administration (empty bars) of a biocompatible polymer, the y-axis representing the time in seconds.

FIG. 4 represents a diagram of the experimental protocol aimed at studying the effects of a co-administration of a biocompatible polymer and of adult stem cells (mesenchymal stem cells) via an MRI study combined with behavioral tests, namely BWT (beam walking test); LP (limb placing test); NS (neurological score) and PA (passive avoidance).

FIG. 5 represents photographs of the central nervous system (brain) (FIG. 5 A) representing an infarction (area within dashed lines) without application (1) or after application (2) of a biocompatible polymer, after application of mesenchymal stem cells (3) and after application of a biocompatible polymer and of mesenchymal stem cells (4) at two days (D2) or fourteen days (D14) following the lesion-inducing ischemic event. FIG. 5 B represents a diagram representing the volume of the lesion (y-axis) as a function of the day (x-axis) without (white bars) or with application of a biocompatible polymer (black bars), with application of mesenchymal stem cells (horizontally hashed bars) or after application of a biocompatible polymer and of mesenchymal stem cells (diagonally hashed bars).

FIG. 6 represents a diagram (FIG. 6 A) representing the results of the limb placing test (*repeated measure ANOVA p<0.05) as a function of the time after administration (solid squares) or non-administration (empty triangles) of a biocompatible polymer, after administration of mesenchymal cells (solid circles) and of a biocompatible polymer and of mesenchymal cells (hashed squares). FIG. 6 B represents a bar diagram of the results of lateralization evaluated using the corner test (* comparison of the mean to the reference value 0 p<0.05) at more or less three days after administration (solid bars) or non-administration (empty bars) of a biocompatible polymer, administration of mesenchymal stem cells and administration of mesenchymal stem cells and of a biocompatible polymer. FIG. 6 C represents a bar diagram of the evaluation of the fine sensorimotor recovery using the adhesive withdrawal test (*p<0.05, one-way ANOVA) after 2 or 4 weeks after administration (solid bars) or non-administration (empty bars) of a biocompatible polymer, with application of mesenchymal stem cells (horizontally hashed bars) or after application of a biocompatible polymer and of mesenchymal stem cells (diagonally hashed bars), the y-axis representing the time in seconds.

FIG. 7 represents optical microscopy photographs of the vascularization in the ischemic area 35 days after occlusion of the middle cerebral artery in the carrier/carrier (A), carrier/mesenchymal stem cells (B), biocompatible polymer/carrier (C) and biocompatible polymer/mesenchymal stem cells (D) groups. The scale is 500 μm.

### EXAMPLES

### Example 1: Evaluation of the Effect of a Biocompatible Polymer According to Some Embodiments on Brain Damage and Functional Deficits Caused by Cerebral Ischemia

In this example, the biocompatible polymer was the polymer sold by the company OTR3 under the trade reference OTR 4131, described in Frescaline G. et al., Tissue Eng Part A. 2013 July; 19(13-14):1641-53. doi: 10.1089/ten.TEA.2012.0377, which is commercially available.

The rats were male rats of the Sprague Dawley strain.

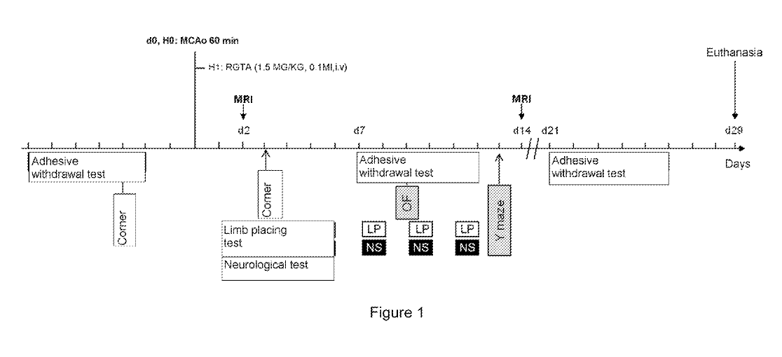

In order to define the effects of the OTR 4131 biocompatible polymer on brain damage and functional deficits, the experimental protocol illustrated in FIG. 1 was carried out in rats subjected to transient cerebral ischemia by occlusion of the middle cerebral artery.

In particular, the animal was anesthetized by inhalation of 5% isoflurane in an O₂/N₂O mixture in respective proportions of 1/3 for 3 minutes, then maintained using 2-2.5% of isoflurane delivered by way of a mask for the time of the surgery. The rat was placed lying down on its back. An incision was made at the level of the neck. The common carotid, external carotid and internal carotid arteries were isolated and then an occlusive wire was introduced into the external carotid and was advanced up to the proximal part of the middle cerebral artery. One hour later, the wire was removed so as to allow reperfusion, for example as described in Letourneur et al., 2011 “Impact of genetic and renovascular chronic arterial hypertension on the acute spatiotemporal evolution of the ischemic penumbra: a sequential study with MRI in the rat” J Cereb Blood Flow Metab. 2011 February; 31(2):504-13 or else Quittet et al., “Effects of mesenchymal stem cell therapy, in association with pharmacologically active microcarriers releasing VEGF, in an ischaemic stroke model in the rat.” Acta Biomater. 2015 March; 15:77-88.

One hour after the induction of ischemia, 1.5 mg/kg of OTR 4131 were administered intravenously, and the animal was then woken up.

In order to evaluate the effects of the treatment on the ischemic volume, an MRI (magnetic resonance imaging (7T, PharmaScan, Bruker BioSpin, Ettlingen, Germany)) study was carried out at 48 h and at 14 days after the induction of the cerebral ischemia. To do this, the animal was anesthetized by inhalation of 5% isoflurane in a 1/3 O₂/N₂O mixture for 3 minutes and then kept anesthetized with 2-2.5% of isoflurane. An anatomical T2 sequence was used according to a RARE 8 rapid acquisition mode with refocused echoes with a repetition time of 5000 milliseconds, an echo time of 16.25 milliseconds, an averaging (NEX number of experiments)=2, a matrix of 256×256 pixels and an image size or FOV (field of view) of 3.84×3.84 cm, that is to say a nominal resolution of 0.15×0.15×0.75 mm³. Twenty contiguous sections were performed per animal with a total acquisition time of 4 minutes.

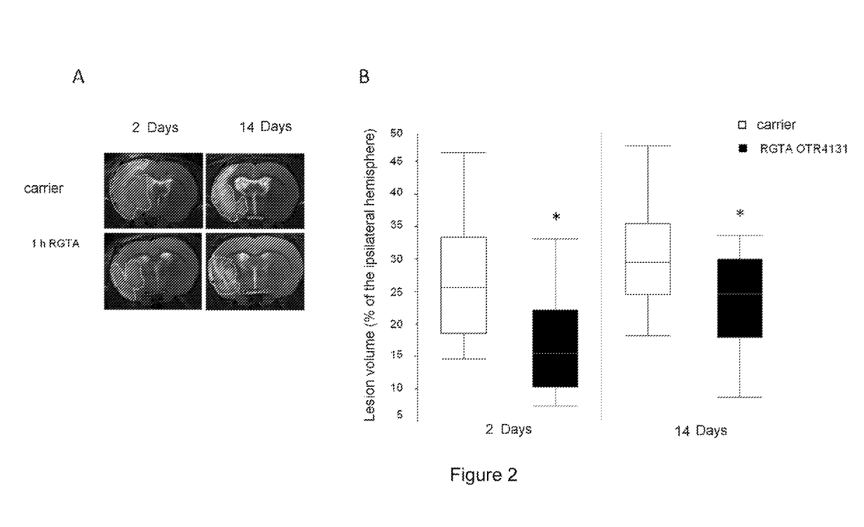

FIG. 2A represents the MRI images obtained after 2 or 14 days after transient cerebral ischemia. As demonstrated in this figure, a decrease in the infarction was observed after an injection, 1 hour after the beginning of the ischemia, of the biocompatible polymer (area surrounded by dashed line) compared with the rat that did not receive biocompatible polymer. A significant decrease in the infarct volume is observed at D2 and at D14 when the treated is administered 1 h post-occlusion (FIG. 2).

This experiment was also carried out while changing the injection time: injection at 2 h30 or at 6 h after the induction of the cerebral ischemia, and showed an absence of significant results (results not provided). In other words, a single injection of the biocompatible polymer 2 h30 or 6 h after ischemia induction does not have any effect on the infarction caused by the ischemia.

An evaluation of the effect of the biocompatible polymer on functional recovery was also carried out. To do this, a battery of sensorimotor and cognitive tests was carried out according to the method described in Quittet et al., “Effects of mesenchymal stem cell therapy, in association with pharmacologically active microcarriers releasing VEGF, in an ischaemic stroke model in the rat.” Acta Biomater. 2015 March; 15:77-88 or Freret et al., 2006 “Long-term functional outcome following transient middle cerebral artery occlusion in the rat: correlation between brain damage and behavioral impairment.” Behav Neurosci. 2006 December; 120(6):1285-98.

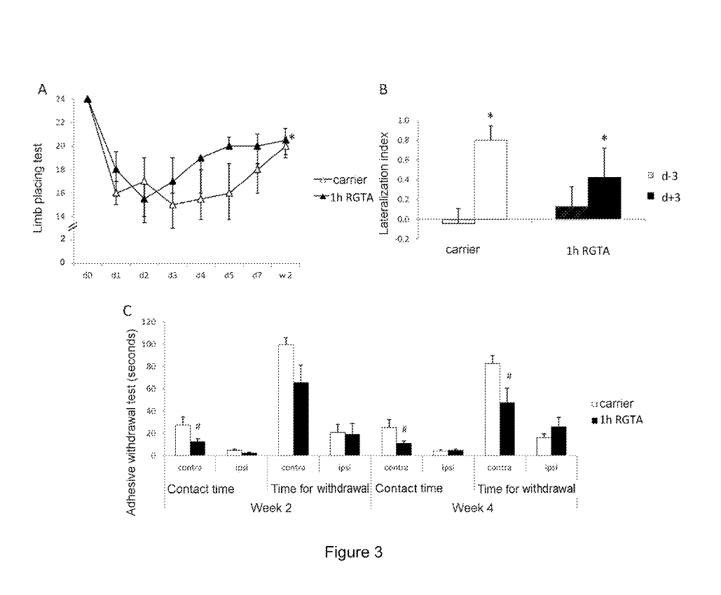

The results obtained are represented in FIG. 3. As demonstrated in this figure, the injection of the biocompatible polymer 1 h after the induction of the ischemia allows an improvement in functional recovery, for example as demonstrated in the limb placing test, evaluating sensory performance (repeated measure ANOVA p<0.05) (FIG. 3A solid triangle) compared with the rat that did not receive an injection, but also in the corner test evaluating the lateralization of the animals via the comparison of the mean to the reference value, p>0.05 (FIG. 3 B solid bars) compared with the rat that did not receive an injection.

In addition, a later fine evaluation of the sensorimotor performance was carried out by way of the adhesive withdrawal test. The results obtained are illustrated in FIG. 3 C. As demonstrated in this figure, the animals that received an administration of biocompatible polymer 1 h post-occlusion (solid bars) have a tendency to detect the presence of the adhesive on the contralesional side affected by the ischemia more rapidly than the other groups that did not receive biocompatible polymer at week 2 (one-way ANOVA, p=0.1). The repetition of the test at week 4 demonstrated a durability of the tendency regarding the detection of the adhesive on the contralesional site (one-way ANOVA, p=0.1). Added to the latter is a significantly faster withdrawal of the adhesive on the contralesional side for the animals treated with the biocompatible polymer, attesting to a more rapid improvement in sensorimotor performance induced by the biocompatible polymer.

### Example 2: Evaluation of the Effect of the Co-Administration of a Biocompatible Polymer and of a Mesenchymal Stem Cell on Brain Damage and Functional Deficits Caused by Ischemic Shock

In this example, the rats and the biocompatible polymer were identical to those of example 1.

The mesenchymal stem cells were extracted from the femurs and tibia of Sprague Dawley rats according to the method described in the document Quittet et al. “Effects of mesenchymal stem cell therapy, in association with pharmacologically active microcarriers releasing VEGF, in an ischaemic stroke model in the rat” Acta Biomater. 2015 March; 15:77-88.

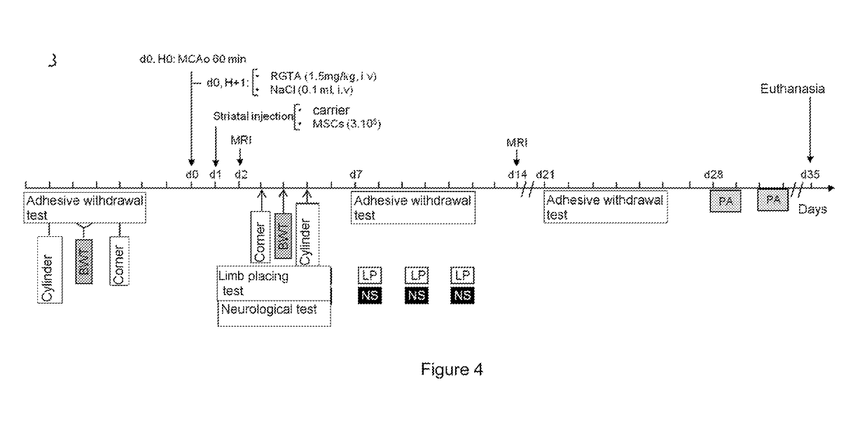

In order to define the effects of the co-administration of the OTR 4131 biocompatible polymer and of the mesenchymal stem cells on brain damage and functional deficits, the experimental protocol illustrated in FIG. 4 was carried out in rats subjected to transient cerebral ischemia by occlusion of the middle cerebral artery according to the intraluminal approach as described in example 1 above.

An evaluation of effects of the co-administration on the infarct volume, but also on the post-ischemic functional recovery, was carried out.

In order to evaluate the effects of the treatment on the ischemic volume, an MRI (magnetic resonance imaging) study was carried out at 48 h and at 14 days after the induction of the cerebral ischemia, as described in example 1 above.

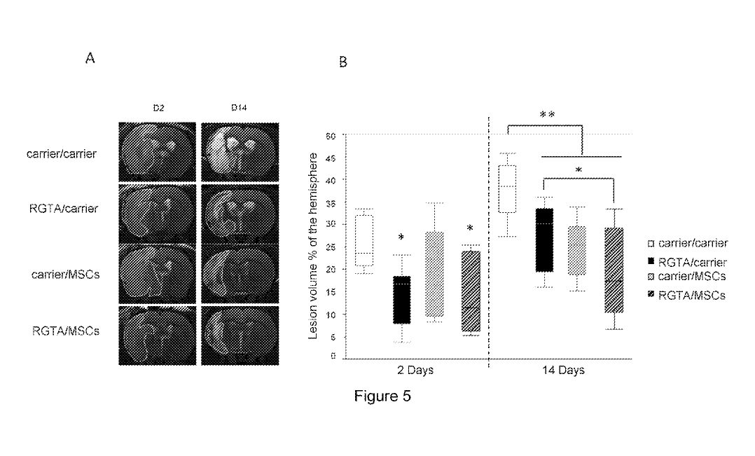

The MRI analysis after 48 hours revealed a decrease in the infarct volume relative to the control group for the animals treated with the RGTA and the RGTA-MSCs co-administration (one-way ANOVA, p<0.05) as illustrated in FIG. 5 A (area within dashed line). In particular, it was clearly demonstrated that the co-administration advantageously enables a decrease in the volume of the lesion at 48 hours compared with the subject that did not receive an injection, but also makes it possible, surprisingly, after 14 days to significantly reduce the volume of the lesion compared in particular with the animals treated with the biocompatible polymer alone or the MSCs alone (FIGS. 5 A and B). Thus, this experiment clearly demonstrates that the composition according to some embodiments and/or the administration of the biocompatible polymer and of the cell makes it possible to obtain a new technical effect not observed in their absence and/or when they are administered alone.

An evaluation of the effect of the co-administration of the biocompatible polymer and of the stem cells on the functional recovery was also carried out. To do this, a battery of sensorimotor and cognitive tests were carried out.

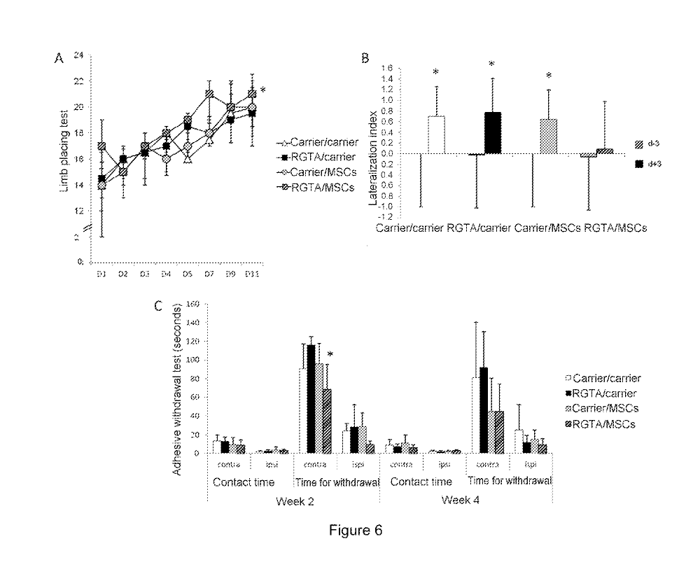

The results obtained are represented in FIG. 6. As demonstrated in this figure, the effects of the treatment on the sensorimotor and cognitive performance, the evaluation of the early sensory recovery by way of the limb placing test (FIG. 6 A) demonstrated a better recovery for the animals of the biocompatible polymer-mesenchymal stem cells group (hashed squares) compared with the other three groups (repeated measure ANOVA p<0.05).

Regarding the lateralization that was evaluated by way of the corner test, a potentiation of the functional recovery was also brought to the fore, the potentiation being demonstrated by the lateralization index not different than the reference value set at 0 (non-lateralization value) only for the animals of the biocompatible polymer-mesenchymal stem cells group (FIG. 6B). Finally, regarding the adhesive withdrawal test, it was demonstrated that the adhesive withdrawal was accelerated starting from the second week post-occlusion in the contralesional side affected by the ischemia only for the animals of the biocompatible polymer-mesenchymal stem cells group (one-way ANOVA p<0.05) (FIG. 6C hashed bars).

These results therefore clearly demonstrate that the combination of the biocompatible polymer and the eukaryotic cells, in particular the mesenchymal stem cells (MSCs), makes it possible to obtain and to treat the tissue lesions of the central nervous system. In particular, it also, surprisingly, enables a much greater functional recovery than that of the non-treated animals, but also one that is much greater than that of the animals treated only with the biocompatible polymer or the MSCs alone.

An ex-vivo evaluation was also carried out. To do this, the sections of the brain were rinsed three times for 5 minutes in 0.1 M PBS and were then incubated in a mixture of 0.1 M PBS/3% BSA (bovine serum albumin, Sigma®)/0.1% triton (Sigma®) for at least 1 hour in order to saturate the nonspecific binding sites. The sections were subsequently placed in contact with the primary antibody (RECA-1; AbDSerotec, diluted to 1:100 in 0.1 M PBS/1% BSA/0.1% triton) overnight at 4′C with gentle stirring. The sections were subsequently rinsed three times with 0.1 M PBS and then incubated for 2 hours with the secondary antibody diluted in a solution of 0.1 M PBS/1% BSA/0.1% triton. The sections were rinsed three times in PBS, before being mounted between slide and coverslip. Photos were acquired using an upright microscope equipped with a camera and with the MetaVue software. The images thus obtained were analyzed using the ImageJ software (http://imagej.nih.gov/ij/).

Thus, the vascularization of the tissue rendered ischemic was evaluated by immunofluorescence using labelling of the endothelial cells with the RECA-1 antibody (Rat Endothelial Cell Antibody-1). The labelling made it possible, where appropriate, to identify and to bring to the fore the vascular architecture of the tissue represented by the white lines in the shaded areas.

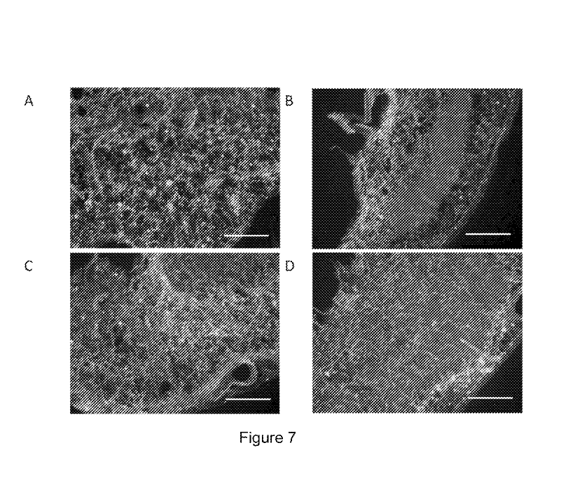

As demonstrated in FIG. 7 representing the electron microscopy photographs obtained, the labelling reveals that, in the absence of biocompatible polymer or of the combination of the biocompatible polymer and of the mesenchymal stem cells, no preservation of the architecture of the vascularization in the infarct zone was observed (FIGS. 7 A and C). Only in the presence of biocompatible polymer (FIG. 7 B) or of the combination of the biocompatible polymer and of the mesenchymal stem cells (FIG. 7 D) could a preservation of the vascular structure be observed. In addition, FIG. 7 D clearly demonstrates that the combination of the biocompatible polymer and of the mesenchymal stem cells makes it possible to obtain a surprising and unexpected effect on this preservation of the vascular structure.

This example therefore clearly demonstrates that the composition according to some embodiments advantageously makes it possible to prevent and/or treat tissue lesions of the central nervous system caused by cerebral vascular ischemia. In addition, this example clearly demonstrates that the composition according to some embodiments also makes it possible to treat possible functional deficits caused by tissue lesions of the central nervous system. In addition, this example clearly demonstrates that the composition according to some embodiments advantageously makes it possible to decrease the recovery time and/or to enable recovery from the possible functional deficits caused by the tissue lesion.

This example therefore clearly demonstrates that the composition according to some embodiments has considerable beneficial effects in ischemia, both in terms of tissue protection, for example by limiting the infarct volume, but also in terms of functional recovery, as illustrated above. Added to these beneficial effects is also an improvement in the preservation of the architecture of the vascular system in the infarct zone.

### LIST OF REFERENCES

1. Adeoye, O., Hornung, R., Khatri, P., & Kleindorfer, D. (2011). Recombinant tissue-type plasminogen activator use for ischemic stroke in the United States: a doubling of treatment rates over the course of 5 years. Stroke; a Journal of Cerebral Circulation, 42(7), 1952-5.
2. Aggarwal, S., & Pittenger, M. F. (2005). Human mesenchymal stem cells modulate allogeneic immune cell responses. Blood, 105(4), 1815-22.
3. Altman, J., & Das, G. D. (1965). Autoradiographic and histological evidence of postnatal hippocampal neurogenesis in rats. The Journal of Comparative Neurology, 124(3), 319-35.
4. Andres, R. H., Horie, N., Slikker, W., Keren-Gill, H., Zhan, K., Sun, G., Steinberg, G. K. (2011). Human neural stem cells enhance structural plasticity and axonal transport in the ischaemic brain. Brain, 134(6), 1777-1789.
5. Bang, O. Y., Lee, J. S., Lee, P. H., & Lee, G. (2005). Autologous mesenchymal stem cell transplantation in stroke patients. Annals of Neurology, 57(6), 874-82.
6. Barritault, D., Garcia-Filipe, S., & Zakine, G. (2010). Basement of matrix therapy in regenerative medicine by RGTA®: From fundamental to plastic surgery. Annales de Chirurgie Plastique et Esthetique, 55(5), 413-420.
7. Bhasin, A., Srivastava, M., Kumaran, S., Mohanty, S., Bhatia, R., Bose, S., Airan, B. (2011). Autologous mesenchymal stem cells in chronic stroke. Cerebrovascular Diseases Extra, 1(1), 93-104.
8. Cramer, S. C. (2008). Repairing the human brain after stroke: I. Mechanisms of spontaneous recovery. Annals of Neurology, 63(3), 272-287.
9. Crisan, M., Yap, S., Casteilla, L., Chen, C. W., Corselli, M., Park, T. S., Peault, B. (2008). A perivascular origin for mesenchymal stem cells in multiple human organs. Cell Stem. Cell, 3, 301-313.
10. Da Silva Meirelles, L., Chagastelles, P. C., & Nardi, N. B. (2006). Mesenchymal stem cells reside in virtually all post-natal organs and tissues. Journal of Cell Science, 119, 2204-2213.
11. Desgranges, P., Barbaud, C., Caruelle, J. P., Barritault, D., & Gautron, J. (1999). A substituted dextran enhances muscle fiber survival and regeneration in ischemic and denervated rat EDL muscle. The FASEB Journal, 13(6), 761-766.
12. Di Nicola, M. (2002). Human bone marrow stromal cells suppress T-lymphocyte proliferation induced by cellular or nonspecific mitogenic stimuli. Blood, 99(10), 3838-3843.
13. Eckert, M. a, Vu, Q., Xie, K., Yu, J., Liao, W., Cramer, S. C., & Zhao, W. (2013). Evidence for high translational potential of mesenchymal stromal cell therapy to improve recovery from ischemic stroke. Journal of Cerebral Blood Flow and Metabolism, 33(9), 1322-34.
14. Erices, A., Conget, P., & Minguell, J. J. (2000). Mesenchymal progenitor cells in human umbilical cord blood. British Journal of Haematology, 109(1), 235-42.
15. Esneault, E., Pacary, E., Eddi, D., Freret, T., Tixier, E., Toutain, J., . . . Bernaudin, M. (2008). Combined therapeutic strategy using erythropoietin and mesenchymal stem cells potentiates neurogenesis after transient focal cerebral ischemia in rats. Journal of Cerebral Blood Flow and Metabolism 28(9), 1552-63.
16. Friedenstein, A., Chailakhjan, R., & Lalykina, K. (1970). The development of fibroblast colonies in monolayer cultures of guinea. Cell Proliferation, 3(4), 393-403.
17. Gage, F. H. (2002). Neurogenesis in the adult brain. The Journal of Neuroscience: The Official Journal of the Society for Neuroscience, 22(3), 612-3.
18. In't Anker, P. S., Scherjon, S. A., Kleijburg-van der Keur, C., de Groot-Swings, G. M. J. S., Claas, F. H. J., Fibbe, W. E., & Kanhai, H. H. H. (2004). Isolation of mesenchymal stem cells of fetal or maternal origin from human placenta. Stem Cells, 22(7), 1338-45.
19. Jaillard, A., Naegele, B., Trabucco-Miguel, S., LeBas, J. F., & Hommel, M. (2009). Hidden dysfunctioning in subacute stroke. Stroke, 40(7), 2473-9.
20. Kranz, A., Wagner, D. C., Kamprad, M., Scholz, M., Schmidt, U. R., Nitzsche, F., Boltze, J. (2010). Transplantation of placenta-derived mesenchymal stromal cells upon experimental stroke in rats. Brain Research, 1315, 128-136.
21. Lee, J. S., Hong, J. M., Moon, G. J., Lee, P. H., Ahn, Y. H., & Bang, 0. Y. (2010). A long-term follow-up study of intravenous autologous mesenchymal stem cell transplantation in patients with ischemic stroke. Stem Cells, 28(6), 1099-1106.
22. Lees, K. R., Bluhmki, E., von Kummer, R., Brott, T. G., Toni, D., Grotta, J. C., Hacke, W. (2010). Time to treatment with intravenous alteplase and outcome in stroke: an updated pooled analysis of ECASS, ATLANTIS, NINDS, and EPITHET trials. The Lancet, 375(9727), 1695-1703.
23. Malgieri, A., Kantzari, E., Patrizi, M. P., & Gambardella, S. (2010). Bone marrow and umbilical cord blood human mesenchymal stem cells: state of the art. International Journal of Clinical and Experimental Medicine, 3(4), 248-69.
24. Paul, G., & Anisimov, S. V. (2013). The secretome of mesenchymal stem cells: Potential implications for neuroregeneration. Biochimie, 95(12), 2246-2256.
25. Rouet, V., Hamma-Kourbali, Y., Petit, E., Panagopoulou, P., Katsoris, P., Barritault, D., Courty, J. (2005). A synthetic glycosaminoglycan mimetic binds vascular endothelial growth factor and modulates angiogenesis. The Journal of Biological Chemistry, 280(38), 32792-32800.
26. Seri, B., Herrera, D. G., Gritti, A., Ferron, S., Collado, L., Vescovi, A., . . . Alvarez-Buylla, A. (2006). Composition and organization of the SCZ: a large germinal layer containing neural stem cells in the adult mammalian brain. Cerebral Cortex, 16 Suppl 1, i103-i111.
27. Song, H., Cha, M.-J., Song, B.-W., Kim, I.-K., Chang, W., Lim, S., . . . Hwang, K.-C. (2010). Reactive oxygen species inhibit adhesion of mesenchymal stem cells implanted into ischemic myocardium via interference of focal adhesion complex. Stem Cells, 28(3), 555-63.
28. Song, H., Song, B.-W., Cha, M.-J., Choi, I.-G., & Hwang, K.-C. (2010). Modification of mesenchymal stem cells for cardiac regeneration. Expert Opinion on Biological Therapy, 10(3), 309-19.
29. Suárez-Monteagudo, C., Hernández-Ramírez, P., Alvarez-González, L., García-Maeso, I., de la Cuétara-Bernal, K., Castillo-Díaz, L., . . . Bergado, J. (2009). Autologous bone marrow stem cell neurotransplantation in stroke patients. An open study. Restorative Neurology 27(3), 151-161.
30. Toma, C., Pittenger, M. F., Cahill, K. S., Byrne, B. J., & Kessler, P. D. (2002). Human mesenchymal stem cells differentiate to a cardiomyocyte phenotype in the adult murine heart. Circulation, 105(1), 93-98.
31. Wang, Y., Liu, J., Tan, X., Li, G., Gao, Y., Liu, X., Li, Y. (2013). Induced pluripotent stem cells from human hair follicle mesenchymal stem cells. Stem Cell Reviews, 9(4), 451-60.
32. Yalvac, M. E., Rizvanov, A. A., Kilic, E., Sahin, F., Mukhamedyarov, M. A., Islamov, R. R., & Palotás, A. (2009). Potential role of dental stem cells in the cellular therapy of cerebral ischemia. Current Pharmaceutical Design, 15(33), 3908-16.
33. Yamagata, M., Yamamoto, A., Kako, E., Kaneko, N., Matsubara, K., Sakai, K., Ueda, M. (2013). Human dental pulp-derived stem cells protect against hypoxic-ischemic brain injury in neonatal mice. Stroke; a Journal of Cerebral Circulation, 44(2), 551-4.
34. Yamauchi, H., Desgranges, P., Lecerf, L., Papy-Garcia, D., Tournaire, M. C., Moczar, M., Barritault, D. (2000). New agents for the treatment of infarcted myocardium. FASEB Journal: Official Publication of the Federation of American Societies for Experimental Biology, 14(14), 2133-4.

1. A pharmaceutical composition for the application as a medicament for the prevention and/or treatment of tissue lesions of the central nervous system caused by a hypoxic cerebral pathological condition, the composition comprising
a biocompatible polymer of general formula (I) below
AaXxYy (I)
wherein:
A represents a monomer,
X represents an R1COOR2 or —R9(C═O)R10 group,
Y represents an O- or N-sulfonate group which corresponds to one of the following formulae —R3OSO3R4, R5NSO3R6, —R7SO3R8, wherein:
R1, R3, R5 and R9 independently represent an aliphatic hydrocarbon-based chain which is optionally branched and/or unsaturated and which optionally contains one or more aromatic rings,
R2, R4, R6 and R8 independently represent a hydrogen atom or a cation M+, and
R7 and R10 independently represent a bond, or an aliphatic hydrocarbon-based chain which is optionally branched and/or unsaturated,
a represents the number of monomers,
x represents the degree of substitution of the monomers A by groups X,
y represents the degree of substitution of the monomers A by groups Y, and
a eukaryotic cell.

2. The composition as claimed in claim 1, wherein the monomers A, which may be identical or different, are chosen from sugars, esters, alcohols, amino acids, nucleotides, nucleic acids or proteins.

3. The composition for the use as claimed in claim 1, wherein the number of monomers “a” is such that the mass of the polymers of formula (I) is greater than 2000 daltons.

4. The composition for the use as claimed in claim 1, wherein x is between 20 and 150%.

5. The composition for the use as claimed in claim 1, wherein the degree of substitution “y” is between 30% and 150%.

6. The composition for the use as claimed in claim 1, wherein the biocompatible polymer also includes functional chemical groups Z, different than X and Y, capable of conferring additional biological or physicochemical properties on the polymer.

7. The composition for the use as claimed in claim 6, wherein the degree of substitution of all of the monomers A by groups Z represented by “z” is from 0% to 50%.

8. The composition for the use as claimed in claim 6, wherein the group Z is a substance capable of conferring better solubility or lipophilicity on the polymers.

9. The composition for the use as claimed in claim 6, wherein the groups Z are identical or different and are chosen from the group including amino acids, fatty acids, fatty alcohols, ceramides, or derivatives thereof, or else targeting nucleotide sequences.

10. The composition for the use as claimed in claim 6, wherein the eukaryotic cell is chosen from the group comprising adult or embryonic eukaryotic cells, bone marrow cells and adipose tissue cells.

11. The composition for the use as claimed in claim 6, wherein the biopolymer is administered in the treatment of tissue lesions of the central nervous system caused by cerebral vascular ischemia:
intravenously or intramuscularly at a dose of from 0.1 to 5 mg/kg of body weight, or
orally in 2 to 5 equal intakes per day in an amount of a daily total of from 15 to 500 mg,
intracranially at a dose of from 0.001 to 1 mg·ml-1,
sublingually before heating as a concentrated aqueous solution of from 1 to 100 mg/ml,
aerially by spraying of a solution comprising from 0.1 to 5 mg/kg of body weight of the polymer,
and wherein the eukaryotic cell is used in the treatment by injection within a period of from 5 minutes to 1 month after the first administration of the biocompatible polymer.

12. A pharmaceutical kit intended to be used for the prevention and/or the treatment of tissue lesions of the central nervous system caused by cerebral vascular ischemia, comprising:
i. a biocompatible polymer of general formula (I) below
AaXxYy (I)
wherein:
A represents a monomer,
X represents an R1COOR2 or —R9(C═O)R10 group,
Y represents an O- or N-sulfonate group which corresponds to one of the following formulae —R3OSO3R4, R5NSO3R6, —R7SO3R8, wherein:
R1, R3, R5 and R9 independently represent an aliphatic hydrocarbon-based chain which is optionally branched and/or unsaturated and which optionally contains one or more aromatic rings,
R2, R4, R6 and R8 independently represent a hydrogen atom or a cation M+, and
R7 and R10 independently represent a bond, or an aliphatic hydrocarbon-based chain which is optionally branched and/or unsaturated,
a represents the number of monomers,
x represents the degree of substitution of the monomers A by groups X,
y represents the degree of substitution of the monomers A by groups Y, and
ii. a eukaryotic cell.

13. The kit intended to be used as claimed in claim 12, wherein the biocompatible polymer is administered
intravenously or intramuscularly at a dose of from 0.1 to 5 mg/kg of body weight, or
orally in 2 to 5 equal intakes per day in an amount of a daily total of from 15 to 500 mg,
intracranially at a dose of from 0.001 to 1 mg·ml-1,
sublingually before heating as a concentrated aqueous solution of from 1 to 100 mg/ml,
aerially by spraying of a solution comprising from 0.1 to 5 mg/kg of body weight of the polymer,
and wherein the eukaryotic cell can be used for injection within a period of from 5 minutes to 1 month after the first administration of the biocompatible polymer.

14. The pharmaceutical kit intended to be used as claimed in claim 12, wherein the biocompatible polymer and/or cell are administered over a period of from 1 day to 3 months.

15. The pharmaceutical kit intended to be used as claimed in claim 12, wherein the biocompatible polymer and/or the cell wherein the administration is daily, twice-daily or weekly.

16. The use of a pharmaceutical composition comprising:
a biocompatible polymer of general formula (I) below
AaXxYy (I)
wherein:
A represents a monomer,
X represents an R1COOR2 or —R9(C═O)R10 group,
Y represents an O- or N-sulfonate group which corresponds to one of the following formulae —R3OSO3R4, R5NSO3R6, —R7SO3R8, wherein:
R1, R3, R5 and R9 independently represent an aliphatic hydrocarbon-based chain which is optionally branched and/or unsaturated and which optionally contains one or more aromatic rings,
R2, R4, R6 and R8 independently represent a hydrogen atom or a cation M+, and
R7 and R10 independently represent a bond, or an aliphatic hydrocarbon-based chain which is optionally branched and/or unsaturated,
a represents the number of monomers,
x represents the degree of substitution of the monomers A by groups X,
y represents the degree of substitution of the monomers A by groups Y, and
a eukaryotic cell,
for producing a medicament for the treatment of tissue lesions of the central nervous system caused by a cerebral hypoxic pathological condition.
